(19) United States
(12) Patent Application Publication
(10) Pub. No.: US 2015/0317913 A1
(43) Pub. Date: Nov. 5, 2015

(54) Optimizing Messages Sent to Diabetic Patients in an Interactive Reporting and Messaging System

(71) Applicant: EosHealth, Inc., Houston, TX (US)

(72) Inventor: Kimon J. Angelides, Houston, TX (US)

(21) Appl. No.: 14/307,906

(22) Filed: Jun. 18, 2014

Related U.S. Application Data
(60) Provisional application No. 61/988,179, filed on May 3, 2014.
(60) Provisional application No. 61/993,373, filed on May 15, 2014.

(51) Int. Cl. G09B 19/00; A61B 5/11; A61B 5/145; G09B 5/00

(52) U.S. Cl. CPC G09B 19/0092; G09B 5/00; A61B 5/1118; A61B 5/14532

(57) ABSTRACT

Disclosed is a system of education, monitoring and advising on glucose testing, diet, exercise and drug administration using a device which is carried by the patient and which is capable of: blood glucose testing, displaying messages advising the patient to initiate blood glucose testing, and of recording the results of the test; of displaying advice or further queries based on analysis of the results, and displaying messages relating to advice, education and/or or further queries based on the analysis. The messages are optimized based on their effectiveness in bringing about a favorable response in the patient's blood glucose level or based on other clinical endpoints.

### RELATED APPLICATIONS

This application claims priority to U.S. Provisional No. 61/988,179, filed May 3, 2014 and to U.S. Provisional No. 61/993,373, filed May 15, 2014.

### FIELD OF THE INVENTION

The field is interactive patient management networks where on receipt of health parameter data from a patient, the network sends the patient particular directives which have increased probability of motivating the patient to take positive action.

### BACKGROUND

As one of America's deadliest diseases, and as there are over 20 million American diabetics, diabetes mellitus places a particularly high expense burden on the public healthcare system. Millions of Americans are not even aware that they have the disease, and an additional 50 million plus Americans have pre-diabetes. If the present trends continues, 1 in 3 Americans, including as many as 1 in 2 minorities born in 2000 will develop diabetes during their lifetime.

Diabetes is a group of chronic metabolic diseases marked by high levels of blood glucose resulting from defects in insulin production, insulin action, or both. While diabetes can lead to serious complications and premature death, effective treatment requires the diabetic patient to take steps to control the disease and lower the risk of complications.

About 5-10% of diabetics have Type I diabetes, while 90-95% have Type 2 diabetes. Type I is an autoimmune disease while Type II results from insulin resistance or inadequate insulin production. Type I has clear genetic markers while Type II is genetically heterogenous and therefore has a broader and less certain origin. Type II diabetes develops later in life, usually as organs & tissues lose their ability to respond effectively to insulin. Risk factors for Type II diabetes include older age, obesity, family history of diabetes, prior history of gestational diabetes, impaired glucose tolerance, physical inactivity, and race/ethnicity. As was mentioned above, African Americans, Hispanic/Latino Americans, American Indians, and some Asian Americans and Pacific Islanders are at particularly high risk for Type II diabetes.

The estimated cost of treatment totals 98 million dollars annually in the US. This problem is compounded by the fact that adult-onset diabetes is increasing at an alarming rate, and also striking at younger ages. Type II diabetes is showing up in young adults and even children. The disease often causes permanent damage to younger victims before they are diagnosed.

Uncontrolled diabetes leads to chronic end-stage organ disease and in the United States is a leading cause of end-stage renal disease, blindness, non-traumatic amputation, and cardiovascular disease. It is also associated with complications such as:

Heart Disease and Stroke (#1 cause of death for diabetics and 2-4 time higher than the general population)

High Blood Pressure (3 in 4 diabetics)

Nervous System Damage (can lead to amputations and carpel tunnel syndrome)

Pregnancy Complications (including gestational diabetes)

Sexual Dysfunction (double the incidence of erectile dysfunction)

Periodontal Disease

In the USA, over 85% of people aged 65 and over have diabetes, a fact that complicates their total health picture and often accelerates chronic end-stage disease, adding an enormous strain to the healthcare system. In addition, there are correlations of higher diabetes incidence with smokers, and Alzheimer's patients.

Poor control of blood-glucose in diabetes dramatically increases the risk of heart disease, stroke, amputations, blindness, renal disease and failure, impotence, and many other diseases—better control of blood-glucose levels greatly mitigates these complications. Coupled with proper education, nutrition, maintenance of stable blood-glucose levels, and regular exercise, many Type 1 and 2 diabetics can minimize the effects of the disease.

With the growing problem of diabetes in developed and developing countries comes a growing need for convenient blood glucose monitoring, and convenient methods for analysis and treatment based on the monitoring. Diabetics need to monitor their blood glucose multiple times a day and record this information, which is analyzed, along with other parameters such as quantity of exercise and their diet, and then use the results to determine food intake, adjust the dosage of insulin and/or other therapeutic agent, and to recommended exercise intensity or cessation. Compliance with the monitoring, diet and exercise regimes is a challenge due to their complexity and temptation to avoid the recommended diet, which is low in simple sugars, and the recommended exercise regime.

A hand-held, portable wireless device, linked to and interactive with a server and with personal health monitors for the user, can be used assist in compliance by reminding the patient of the need to test periodically, by logging the blood glucose test results and the associated meal information and the carbohydrates ingested and the patient feelings, (and storing the results in a user friendly display form as averages and other analysis), and also by providing selected advisory and educational messages, and providing sharing with select health monitors and other selected parties, all with the aim to increase compliance with the recommended the monitoring, diet and exercise regimes. Maintaining an optimal diet and exercise program is extremely important but also problematic for most diabetics. Messages regarding diet, exercise and general education and warnings can be helpful to keep a patient on track. A method of optimizing the messages so that the ones which are most effective in motivating the patient to take appropriate action to maintain diet and exercise would be desirable.

### SUMMARY OF THE INVENTION

Disclosed is a process of increasing patient compliance, especially for diabetics, with a recommended diet and exercise regime, by determining which among a group of messages advising the patient about food intake, timing of food intake, ceasing or commencing exercise and messages relating to the benefits or detriments of particular diet and exercise choices, are most effective in causing the patient to undertake the desired conduct. The advisory messages can also include messages advising the patient to test for a chemical or biochemical indicator, including blood glucose level, ketone level, in vivo drug or insulin concentration, blood pressure, or gene expression level, and the effectiveness in motivating such conduct can also be monitored and determined.

The process is used in an interactive system where patient information (which can be initially input and updated constantly), including information about patient medications, scheduling and dosage, personalized information about suitable exercise, foods and medications, as well as contemporaneous information about diet and exertion level, is transmitted wirelessly to a server for analysis and determination of which messages are to be sent to the patient. In one exemplary message selection protocol, the advisory messages are weighted based on their average effectiveness in moving patients to diet and exercise in a manner which moves their blood glucose level into a desired range or maintains their blood glucose levels in a desired range (or, are most effective in moving patients towards said desired range) wherein averaged effectiveness is the effectiveness of particular messages in causing patients to take actions which make their BG levels move into a desired range or which cause users to take actions which maintain their BG levels in a desired range (or, are most effective in moving patients towards said desired range) over the number of times said particular messages are displayed on the patient's device. Averaged effectiveness can also be determined based on trends in the BG level, such that those messages inducing a downward trend over time are considered more effective than those which induce an immediate decline which then trends upwardly over time. The average effectiveness of advisory messages relating to testing for a chemical or biochemical indicator can be separately weighted in accordance with their average effectiveness in prompting the user to test. The weighting then determines how frequently a particular message is sent. Alternatively, messages with less than a specified weight can be withheld, and never sent; or only sent if the previously more effective messages become less effective over time or otherwise.

The most desired range of blood glucose level is 90 to 125 mg/dL. Under 90 mg/dL would be hypoglycemic and a range of 125 to 180 mg/dL would represent initial stages of hyperglycemia. If blood glucose level is over 180 mg/dL it represents hyperglycemia, and at over 250 mg/dL, it is severe hyperglycemia and ketone levels must be monitored and brought back to normal, if outside an acceptable range. Accordingly, blood glucose level can be a determinative in deciding which of several groups of messages are sent—i.e., it can preempt the weighting and average effectiveness when threshold blood glucose levels are exceeded or not met.

In another aspect, the effectiveness of a group of messages directing the patients to monitor BG levels can be optimized based on how frequently patients test their BG levels following receipt of such messages. Similarly, the effectiveness of a group of messages in directing the patient to exercise can be optimized based on results from an accelerometer carried by the patient (which is preferably part of the device) which shows the patient movement and exertion level.

Messages can also be optimized based on: (i) their effectiveness in reducing co-morbidities and physiological risk factors; (ii) their effectiveness in inducing compliance with medication and other prescribed regimes; (iii) their effectiveness in regulating levels of other biometric parameters besides BG levels including HbA1c, and LDL; and (iv) their effectiveness in inducing adherence to good diabetes care practices, like monitoring of eye, foot, wound and heart health. The messages for each of these categories (i) to (iv) would be weighted based on their effectiveness (which could be measured by a number of methods). Effectiveness of combinations of messages could also be determined against combinations of parameters—for example, it might be that messages relating to category (iv) also induce patient compliance with category (ii) parameters. Or, a combination of messages directed to induce compliance with category (ii) an (iv) also increase compliance with category (i).

The effectiveness of optimizing the messages in controlling BG levels can be advertised or publicized to recruit additional patients into the system, and thus increase the number of patients benefitted.

The invention is described further in the flow charts, where exemplary sets of steps to be executed by a computer are set forth.

### BRIEF DESCRIPTION OF THE FIGURES

FIG. 1 is a flow chart showing optimizing messages to be sent to patients based on their BG levels following receipt of particular messages.

FIG. 2 is a flow chart showing optimizing messages for a particular user including accounting for the number of times the message was sent to the user, based on his/her BG levels following receipt of particular messages.

### DETAILED DESCRIPTION

Preferred user devices and interactive systems for use with the invention include those described in U.S. Pat. No. 8,066,640 and US Publ'n No. 20130035563 (both of which are incorporated by reference). In brief, these references together describe a system of education, monitoring and advising on glucose testing, diet, exercise and drug administration using a device which is lightweight and portable (and easily carried by the patient) and which is capable of: blood glucose testing, displaying messages advising the patient to initiate blood glucose testing, and of recording the results of the test; of displaying advice or further queries based on analysis of the results, including advising for testing ketones if the blood glucose level is above a threshold level; analyzing other blood glucose-related and health-related information and personal information, including patient-identifying information and patient preferences (particularly for diet and exercise) which can input by the patient periodically or input and stored; and of displaying advice, education and/or or further queries based on the analysis.

As the device's computing power or access to full patient information is limited, and because the ability of health care professionals to provide advice is also desired, the device is preferably linked wirelessly to a server that performs some or all of the analysis and information storage described above. In the case of employing a server, the glucose test results and preferably also information about food intake, exertion and patient feelings and symptoms, are transmitted to the server. The device receives the results of the server's analysis in the form of queries, advice and educational messages. The wireless link to the device also provides the ability for feedback, advice and/or intervention from appropriately experienced health care workers, as necessary and appropriate.

The device preferably also includes the ability to test ketone levels and record the results, track timing of food consumption and foods, particularly carbohydrates, consumed, and a pedometer or accelerometer to track patient exertion and estimate total calories expended in exercise.

The analysis from the server is then used to select from a library of messages to send to the device (and the user). The messages relate to advice on further testing, food consumption and exertion, as well as general diabetes education, and are preferably suitable for display on a small screen, typical of a hand-held device—meaning the messages are necessarily compact. The messages user's receive are optimized based on their effectiveness, where effectiveness is based on the patient's BG level response for messages relating to diet and exercise and general advice and warnings. The effectiveness based on the patient's BG level response is a clinically relevant reflection of the effectiveness of such messages in motivating users to adhere to the recommended diet and exercise. Effectiveness cold also be based on recognized clinical endpoints associated with diabetes.

For message which prompt the user to test BG levels or other indicators, effectiveness can be based on the lag time to the next BG test (or other test). Effectiveness of messages prompting the user to exercise (or to cease exercise) can be optimized based on the user's exertion level following such messages, as measured by the pedometer on the user's device.

It is noted, however, that in any message optimization system, certain messages are prioritized where the analysis shows that the need for certain messages much outweighs that of others—in the case, for example, of acute conditions. For example, where BG level indicates hyperglycemia (over 180 mg/dL) or severe hyperglycemia (over 250 mg/dL), particular messages, e.g., “inject insulin” “commence exercise” “check ketones” “don't eat” should be preferentially selected, as the patient is in an acute state. Similarly, certain messages should be prioritized when the patient is hypoglycemic or severely hypoglycemic (less than 70 mg/dL; see US Publn No. 20130035563). The messages in the event of severe hypoglycemia are preferably messages instructing on the “rule of 15” described in US Publn No. 20130035563.

Even where certain messages are prioritized, however, the effectiveness of prioritized message sets can be optimized against returning BG levels to normal ranges, or closer to normal ranges, or against other indicators (e.g., ketone levels) or against established clinical endpoints. An example of optimizing prioritized messages is instead of “commence exercise”: “start walking now”; or instead of “don't eat,” the message could be “eat no food for the next _____ hours.”

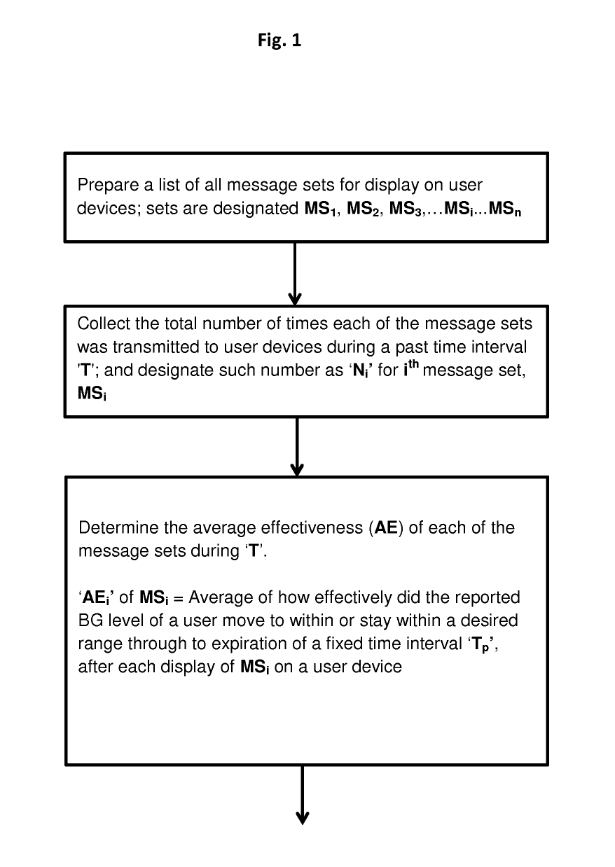

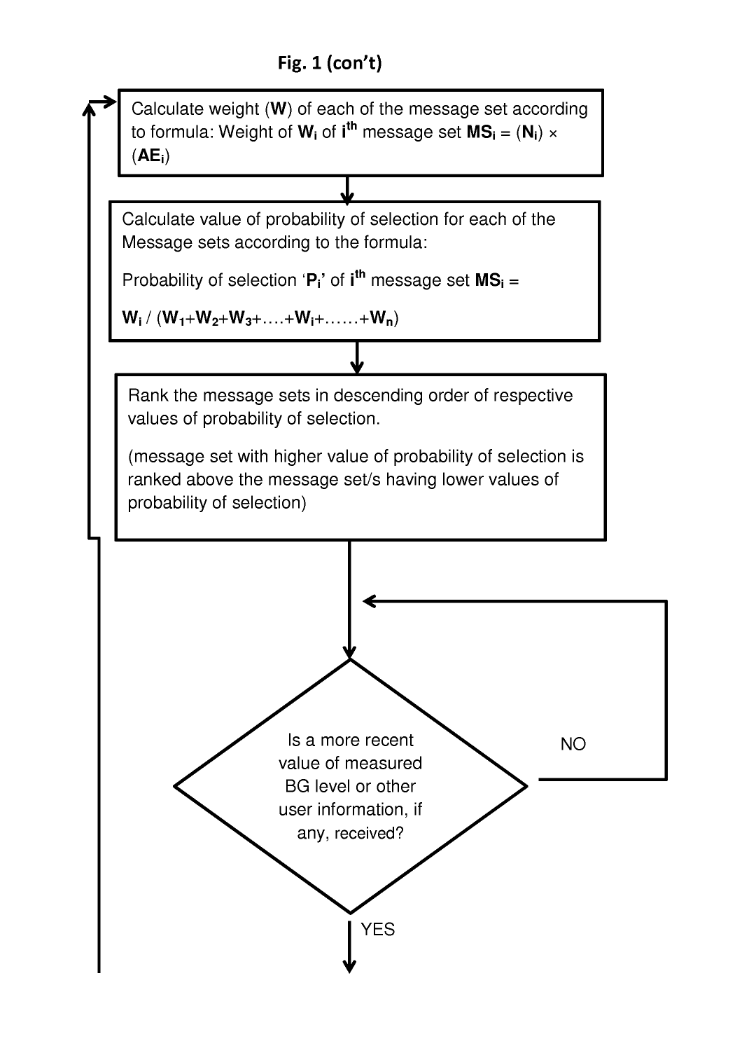

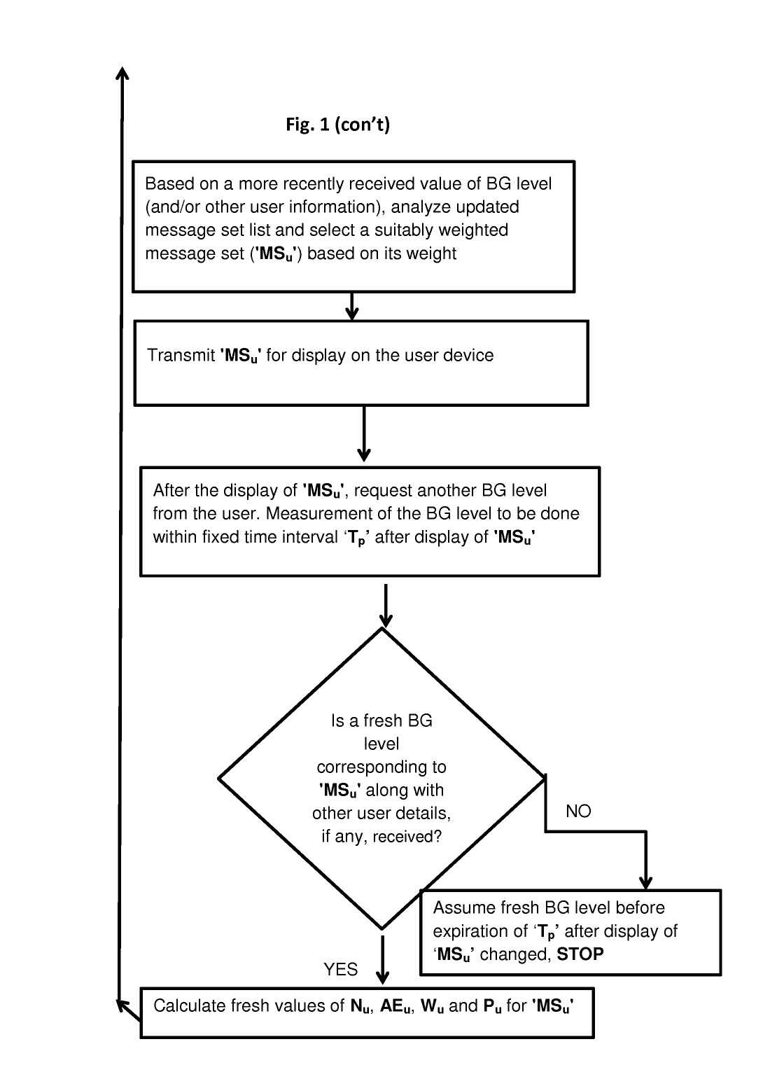

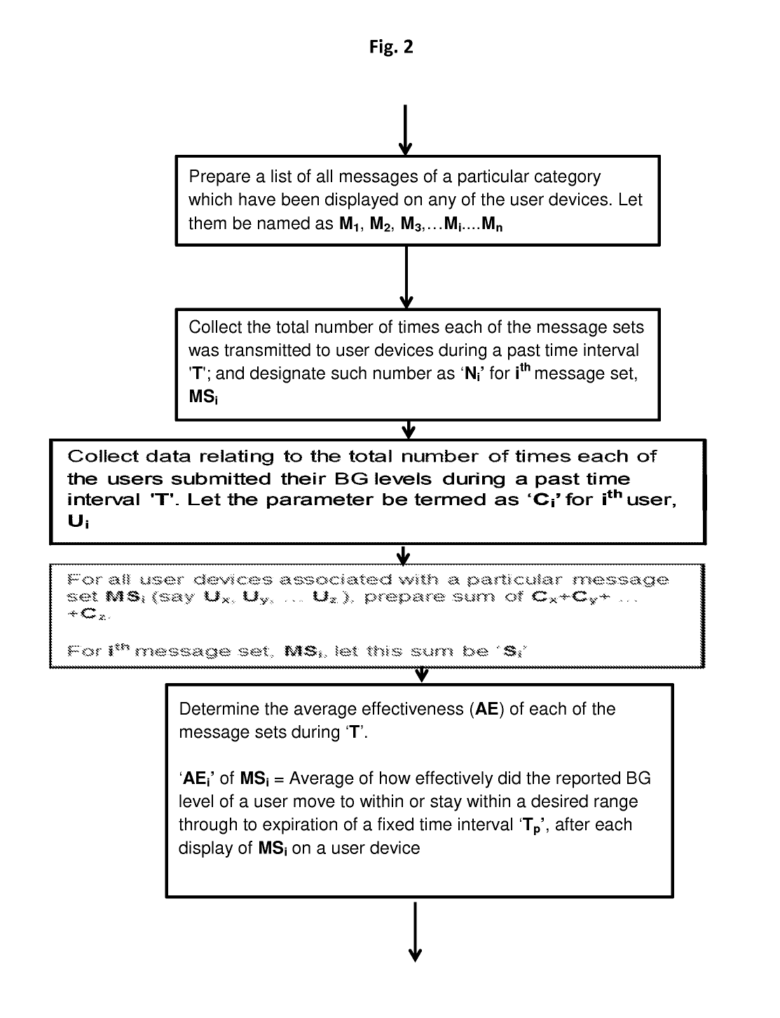

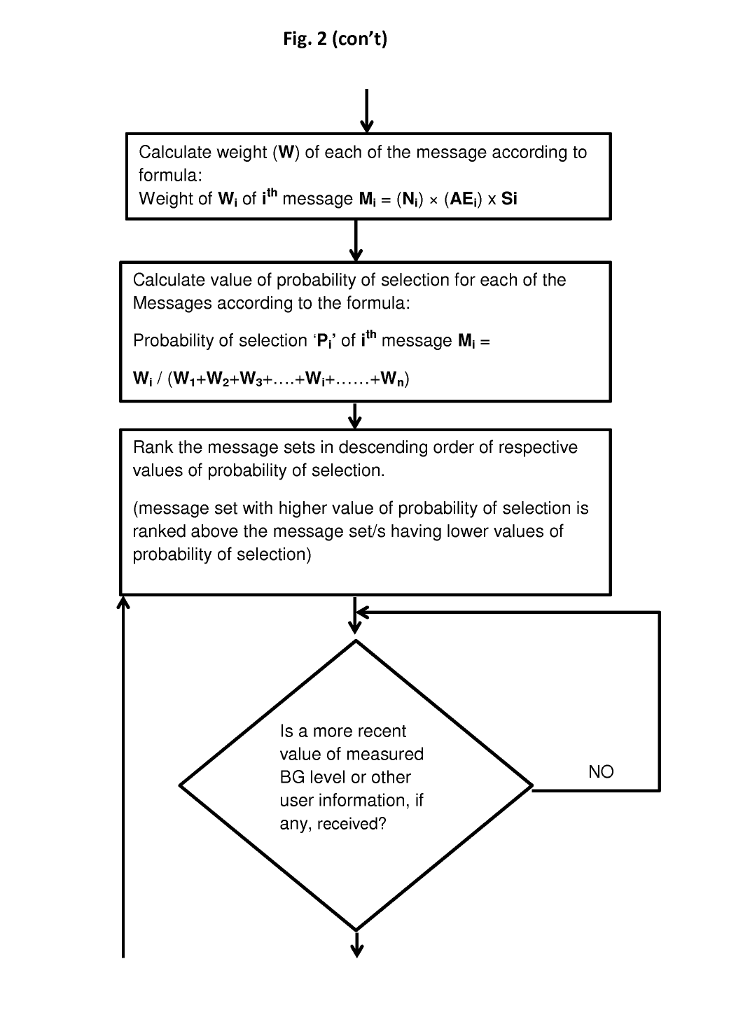

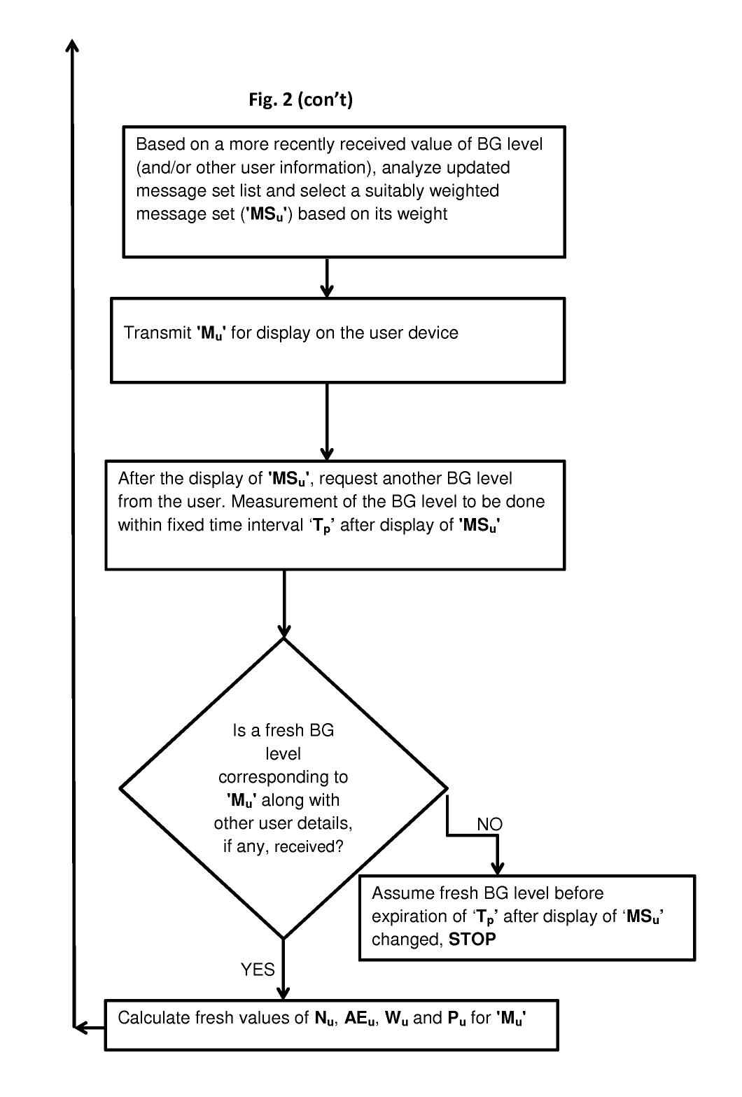

Optimization of messages can be performed a number of ways (i.e., by a number of different algorithms and statistical analysis methods) including by following the steps set forth in FIGS. 1 and 2. The steps outlined in FIGS. 1 and 2 describe a continuous message optimization loop, where the message sets are continuously optimized based on newly received BG levels (provided the BG levels are received within time Tₚ after display of a particular message set Mₙ). Message sets are weighted based on their effectiveness in causing positive changes in the patient's BG level, such that more effective message sets are more frequently selected for display on patients' devices. A similar weighting based on positive effect could be used where another indicator level (e.g., ketone level) or a clinical endpoint is used in determining effectiveness of messages.

If one starts the optimization process with a library of message sets and of BG level responses from patients who received the message sets, then the first cycle through the process of FIGS. 1 and 2 (which follows weighting of more effective messages and preferentially sending them based on their weight) provides an immediate clinical benefit for the patients. Further optimization by continuing the process through subsequent cycles would continuously provide even more effective messages to more patients, to continuously increase the benefit to more patients. Determining whether message sets' effectiveness is statistically significant (i.e., if some sets or orderings or timings of messages improve average BG levels in a statistically significant manner, with a p value of 0.05 or less) would be a further verification of efficacy of such messages. Such determination could also be performed in the system described herein.

In an alternative method where there is no continuous optimization, one could do an initial review of the library and of BG level responses from patients who received the message sets, and select the message sets that were most effective (whether their effectiveness was statistically significant or not)—and send only those message sets subsequently. Similarly, one could run the process for a designated number of cycles and then select the only the most effective sets for sending to patients subsequently. These alternatives limit the ability to include new messages or other changes in message ordering or timing, which may be a disadvantage. Patient responses to optimized messages my change over time, and the ability to test new messages and formats continuously would seemingly be advantageous.

Besides BG level, other clinical endpoints against which message sets can be optimized are death or diabetic disease markers, including non-healing wounds, hypertension, neuropathy, nephropathy, stroke, gastroparesis, ulcers, heart disease, and cataracts. The optimization can be based on the Kaplan-Meier estimator against death or an endpoint associated with any of the foregoing diseases/conditions. In the case where one starts with a library of messages sent to diabetic patients over a prior period and information about whether they reached death or another endpoint associated with any of the foregoing diseases/conditions, these messages can be immediately optimized based on the Kaplan-Meier estimator, and a p value for particular messages or message sets can be derived, by either comparison among patients in the database or against established or known values of progression to the endpoint(s). Messages or message sets that are effective in prolonging reaching an endpoint with a p value of 0.05 or greater, which are those shown to be beneficial in a statistically significant manner, can be designated to be always sent (i.e., be exclusively selected). Alternatively, the most effective messages (whether their effectiveness is statistically significant or not) can be more heavily weighted in subsequent loops of the process where the optimization is a continuous function (as in FIGS. 1 and 2).

Referring to FIGS. 1 and 2, another way to determine average effectiveness of messages (AEᵢ of Mᵢ), rather than to average “how effectively did the reported BG level of a user move to within or stay within a desired range” (as shown), is to determine how much (on average) the messages caused patient BG level to move towards that range. That is, messages which are associated with moving BG level from further out of range to closer to the desired range are more effective and are weighted in accordance with the amount of such movement (or change). See FIG. 3.

Another variation on the process in FIGS. 1 and 2 is to use other math functions besides weighting, including Kaplan-Meier or other regression analysis, to determine average effectiveness. A number of algorithms can be used to optimize messages or message sets.

Although FIGS. 1 and 2 specify ranking “the message sets in descending order of respective values of probability of selection,” this may not be a necessary step—though it can facilitate selection when using software-driven methods of selection.

Once a library of messages is established together with a database of patient responses, the process in FIGS. 1 and 2 can be used to optimize message sets for particular segments of the patient population (where segmenting can be based on, for example, age, sex, education level or ethnicity). The population segment the patient belongs to can be identified from the patient information in the database (note that the patient inputs personal and identifying information and preferences into the server's database).

The patient population could also be segmented based on their preferences, including their diet and exercise preferences. Monitoring of the message library and patient responses can allow such segmenting, as patient preferences are preferably entered into the database on the server, and messages to such patients can then be correlated with effectiveness to optimize them. Patients with preferences for particular foods or exercises, may well be more responsive to certain messages regarding diet and exercise—making optimization for such patient segments desirable.

As noted, the messages can be optimized across the message characteristics, including language choices, punctuation and grammar, font and format. Optimization can be of message sets or individual messages. For individual messages, their ordering and timing of sending them (in relation to each other) can also be optimized, following optimization of the message characteristics. For message sets, the optimization can further include the ordering and the timing of the sending of the different messages in each set, increased frequency of repetition for some messages in a set, and can further include the timing of and the order of sending of different sets in relation to other sets.

Message sets could also be divided into subparts based on whether they relate to prompting diet or exercise, or whether they are general educational content messages. The general educational content messages have greater numbers of possible choices than other messages, and thus a greater number of variable terms. It might be desirable to continue to vary and optimize educational messages (in a message set) after the diet and exercise messages in the set have been optimized and certain ones selected. Certain educational messages could also prioritized along with certain diet and exercise messages which are prioritized—when, for example, BG level is far outside the desired range, as described above. Alternatively, when diet and exercise messages are prioritized, the entire educational message library could still be optimized—i.e., no educational messages are prioritized out of the library.

As noted, the effectiveness of messages prompting exercise can be among those monitored in determining effectiveness in controlling BG levels. Messages prompting exercise can also separately monitored based on the patient's change in exertion level during a specified time following the message display. Where multiple messages or where message sets are sent, the effectiveness can be determined over a longer period—for example, effectiveness in increasing exercise time or intensity over a month-long period can be determined.

For devices including a pedometer, the exertion level is preferably determined by the pedometer and transmitted for analysis. Or, exertion level can be by (or pedometer results can be supplemented by) patient reporting. All the segmenting and message variation applicable to optimizing messages about BG level could also be used to segment (among populations) or vary (including variation of timing of) messages prompting exercise.

The algorithms for determining effectiveness of messages prompting exercise can be similar to those shown in FIGS. 1 and 2—i.e., a continuous loop where the initially more effective messages are weighted and sent more frequently than the less effective ones. Again, rather than a continuous loop it can be preferred to simply select the most effective messages (either from a library of responses or after a certain number of cycles through the loop) and use only those messages (or only those message sets) going forward. Other functions and algorithms for determining effectiveness besides the weighting method in FIGS. 1 and 2 can also be applied to messages prompting exercise.

Messages prompting the user to test BG levels would normally be separately monitored for Effectiveness—based on whether the test was performed within a specified period following sending the message. All the segmenting and message variation applicable to optimizing messages about BG level could also be used to segment (among populations) or vary (including variation of timing of) messages prompting testing.

In FIGS. 1 and 2 it shows that without such a BG level test, there are no results available to determine message effectiveness in moving BG level to the desired range. Without a BG level test in the process shown in FIGS. 1 and 2, the message effectiveness would be that determined solely from library of messages and patient responses. Thus, one variation on the process in FIGS. 1 and 2 is to factor in the number of BG level tests which are used in determining average message effectiveness—in order to increase reliability of the effectiveness determined.

The specific methods, processes and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. The term “messages” includes “message sets.” Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing, by Applicants. The invention has been described broadly and generically herein. Each of the narrower species and subgeneric groupings falling within the generic, disclosure also form part of the invention.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A process of increasing diabetic patient compliance with a recommended diet and exercise regime, comprising:
providing a recommended diet and exercise regimen for the patient to follow for a particular forthcoming period;
providing an interactive wireless link between a server and a device carried by the patient:
(i) wherein the device is actuated by the patient to test a patient blood sample for patient blood glucose level and the device determines patient exertion level by measuring patient movement or acceleration and the device actively sends the determinations of said blood glucose level and exertion level to the server, and where the device or the server actively queries the patient about prior food consumption and time of food consumption,
(ii) wherein the server analyzes the blood glucose level test results, exertion level and query responses, and sends the patient advisory messages about future food consumption and timing of food consumption, about timing of further testing, and also sends the patient advisory messages about commencing, continuing or ceasing exertion, and also sends the patient educational messages about particular diet and exercise choices;
weighting the advisory messages based on their average effectiveness in moving patients to diet and exercise in a manner which moves their blood glucose level into a desired range or maintains their blood glucose levels in a desired range, wherein averaged effectiveness is the effectiveness of particular messages in causing patients to take actions which make their BG levels move into a desired range or which cause users to take actions which maintain their BG levels in a desired range over the number of times said particular messages are displayed on the patient's device;
selecting messages frequency of display on the patient's device in accordance with the respective weight of the selected messages;
repeating the weighting of messages based on their average effectiveness and the selection of messages for display in accordance with the respective weight of the selected messages.

2. The process of claim 1 wherein messages below a certain weight are not sent.

3. The process of claim 1 wherein the repeating step is repeated several times based on the average effectiveness of messages sent.

4. The process of claim 1 wherein the particular messages include message groups, where a message group includes messages regarding food intake, timing of food intake, ceasing or commencing exercise and messages relating to the benefits or detriments of particular diet and exercise choices.

5. The process of claim 4 wherein the particular messages include only message groups about the benefits and detriments of particular diet and exercise choices.

6. The process of claim 1 wherein the desired range of BG level is 90 to 125 mg/dL.

7. The process of claim 1 wherein the desired range of BG level is 90 to 180 mg/dL.

8. The process of claim 1 wherein during the selection process, messages are ranked in order of their weight by the server.

9. The process of claim 1 wherein if the BG level is outside a specified range, the server also selects particular messages for display to the user without regard to their weight.

10. A process of selecting particular messages for display to diabetic patients which are most effective in moving diabetic patients to diet and exercise in a manner which moves their blood glucose level towards a desired range or maintains their blood glucose levels in a desired range, comprising:
providing a recommended diet and exercise regimen for the patient to follow for a particular forthcoming period;
providing an interactive wireless link between a server and a device carried by the patient,
(iii) wherein the device is actuated by the patient to test a patient blood sample for patient blood glucose level and the device actively determines patient exertion level by measuring patient movement or acceleration and the device sends the determinations of said blood glucose level and exertion level to the server, and where the device or the server actively queries the patient about prior food consumption and time of food consumption,
(iv) wherein the server analyzes the blood glucose level test results, exertion level and query responses, and sends the patient advisory messages about future actions including food consumption and timing of food consumption, and about commencing, continuing or ceasing exertion, and also sends the patient educational messages about particular diet and exercise choices;
weighting the advisory messages based on their average effectiveness in moving patients to diet and exercise in a manner which moves their blood glucose level into a desired range or maintains their blood glucose levels in a desired range, wherein: averaged effectiveness is the effectiveness of particular messages in causing patients to take actions which make their BG levels move into a desired range or which cause users to take actions which maintain their BG levels in a desired range over the number of times said particular messages are displayed on the patient's device;
selecting the probability of display of particular messages on the patient's device in accordance with the respective weight of the selected messages such that only messages with greater than a designated average effectiveness are displayed or such that messages having greater weight are displayed more often; and
repeating the last two steps of weighting the advisory messages based on their average effectiveness and of selecting particular messages for display.

11. The process of claim 10 wherein the particular messages selected are combinations of advisory messages about future food consumption and timing of food consumption, about timing of further testing, about commencing, continuing or ceasing exertion, and about the benefits or detriments of particular diet and exercise choices.

12. The process of claim 10 wherein the particular messages selected are advisory messages about the benefits or detriments of particular diet and exercise choices.

13. The process of claim 10 wherein the particular messages selected are sent in a particular sequence and over a particular period.

14. The process of claim 10 wherein the particular messages selected advise taking the same actions as messages not selected.

15. The process of claim 10 wherein the server also sends the patient advisory messages about the timing of further blood glucose level testing.

16. The process of claim 15 wherein average effectiveness of said advisory messages about timing or the frequency of the patient's blood glucose testing is determined.

17. The process of claim 16 wherein particular advisory messages which have the greatest average effectiveness in moving patients to test their blood glucose are sent by the server more frequently than others.

18. The process of claim 16 wherein the advisory messages are weighted based on their average effectiveness in moving patients to test their blood glucose and those messages with the greatest weight are sent more frequently than others.

19. The process of claim 1 wherein if the BG level is outside a specified range, the server also selects particular messages for display to the user without regard to their weight.
